(19) United States
(12) Patent Application Publication
(10) Pub. No.: US 2026/0043768 A1
(43) Pub. Date: Feb. 12, 2026

(54) INTERROGATION AND OUTPUT CORRECTION OF CAPILLARY-LIMITED OXYGEN SENSORS

(71) Applicant: MSA Europe Gmbh, Rapperswill-jona (CH)

(72) Inventor: Sebastian Ross, Bad Münder am Deister (DE)

(21) Appl. No.: 19/137,429

(22) PCT Filed: Nov. 24, 2023
(86) PCT No.: PCT/EP2023/083000

§ 371 Date: Jun. 10, 2025

(30) Foreign Application Priority Data
Dec. 15, 2022 (DE) 102022133531.3

(51) Int. Cl. G01N 27/404; G01N 27/416

(52) U.S. Cl. CPC G01N 27/404; G01N 27/4161

(57) ABSTRACT

A method of operating a capillary-limited electrochemical gas detection device includes applying an electrical signal to the electrochemical sensor of the gas detection device to generate a current flow between a working electrode and a counter electrode of the electrochemical gas sensor via an electrolyte of the electrochemical gas sensor, measuring a parameter of a response of the electrochemical sensor to the electric signal, comparing the measured parameter to a predetermined characterization of the parameter, wherein the predetermined characterization of the parameter provides a relationship between the parameter and a response of the electrochemical sensor over varying state of the electrochemical sensor, and determining an output value of the gas detection device from an output signal electrochemical sensor in sensing oxygen in the ambient environment and the comparison of the measured parameter to the predetermined characterization.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Electrochemical sensors are effective in detecting various gases. The low cost, speed of response and selectivity of electrochemical sensors are just a few of the characteristics that have made such sensors attractive for safety products. However, one of the necessary requirements for their use is frequent calibration with a test gas including a known concentration of the analyte gas. Moreover, gas detection instrumentation including electrochemical gas sensors must be tested regularly for functionality. It is a common practice to, for example, perform a “bump check,” or functionality check on portable gas detection instrumentation on a daily basis. The purpose of this test is to ensure the functionality of the entire gas detection system (including the sensor(s) and transport paths), commonly referred to as an instrument. A periodic bump check or functionality check may also be performed on a permanent gas detection instrument to, for example, extend the period between full calibrations. Gas detection systems include at least one gas sensor, electronic circuitry to drive the sensor, interpret its response and display its response to the user, and a power supply. The systems further include a housing to enclose and protect such components. A bump check typically includes: a) applying a gas of interest (usually a gas having a known concentration of the analyte gas or a simulant therefor); b) collecting and interpreting the sensor response; and c) indicating to the end user the functional state of the system (that is, whether or not the instrument is properly functioning).

A number of systems and methods have been proposed to, for example, reduce the number of periodic tests using test gas in diffusion-limited electrochemical gas sensors while providing a frequent measure of sensor life and health. Such a system may, for example, include electronic interrogation of a sensor in the absence of a test gas. The fluctuation in sensitivity arising from moisture loss or gain in diffusion-limited electrochemical sensors occurs gradually but in a predictable manner as the average relative humidity slowly changes in such sensors. Likewise, the sensor response to an electronic interrogation (in the absence of or without application of a test gas including a known concentration of the analyte gas or a substitute therefor) changes in a similar manner. An electronic interrogation may, for example, be used to measure sensitivity changes and to correct sensor output for such sensitivity changes.

Electronic interrogation techniques and resulting corrections for diffusion-limited electrochemical gas sensors are, for example, disclosed in U.S. Pat. Nos. 7,413,645, 7,959,777, 9,784,755, and 9,528,957, and in U.S. Patent Application Publication Nos. 2013/0186777 and 2017/0219515, the disclosures of which are incorporated herein by reference. In such electronic interrogation approaches, an electrical signal such as a potential pulse is typically applied to the sensor and the resulting response is measured and recorded. Electronic interrogation of capillary-limited gas sensors is described in U.S. Pat. No. 11,112,378, the disclosure of which is incorporated herein by reference.

While electronic interrogation of capillary-limited electrochemical gas sensors for oxygen are failure-mode sensitive, such interrogations have not delivered consistent data to correct for sensor behavior/sensitivity changes in the field. It is thus desirable to develop improvised electronic interrogation technologies for use in connection with capillary-limited electrochemical gas sensors for oxygen.

### SUMMARY

In one aspect, a method of operating a gas detection device (which includes a capillary-limited electrochemical sensor which is analytically responsive to oxygen, wherein the electrochemical sensor includes a housing including a capillary via which gas diffuses into the housing from an environment, a working electrode within the housing, a counter electrode within the housing, and an electrolyte within the housing in ionic contact with the working electrode and the counter electrode) includes applying an electrical signal to the electrochemical sensor to generate a current flow between the working electrode and the counter electrode via the electrolyte, measuring a parameter of a response of the electrochemical sensor to the electric signal, comparing the measured parameter to a predetermined characterization of the parameter, wherein the predetermined characterization of the parameter provides a relationship between the parameter and a response of the electrochemical sensor over varying state of the electrochemical sensor, and determining an output value of the gas detection device from an output signal electrochemical sensor in sensing oxygen in the ambient environment and the comparison of the measured parameter to the predetermined characterization. In a number of embodiments, the method further includes operating the electrochemical sensor in a sensing mode wherein the output signal, which is representative of a concentration of oxygen in the environment, is generated, and operating the electrochemical sensor in an interrogation mode during which the electrochemical sensor is electronically interrogated by applying the electrical signal to the electrochemical sensor, the measured parameter being measure in the interrogation mode.

The gas detection device may further include a control system including a processor system and a memory system, wherein the working electrode is in operative connection with the control system and the counter electrode is in operative connection with the control system. The memory system includes one or more algorithms stored thereof and executable by the processor system to carry out one or more the actions of the method. The predetermined characterization may be saved in memory at the time of manufacture.

In a number of embodiments, the parameter is a chronoamperometric parameter. The parameter may be, or may be a function of, maximum peak value, area under the curve, minimum peak value, peak-to-peak value and reverse area under the curve of the response of the electrochemical sensor to the electric signal applied in the interrogation mode.

The predetermined characterization may be determined over a varying state of the electrochemical sensor. The varying state of the electrochemical sensor may be induced by environmental condition.

In a number of embodiments, the predetermined characterization is determined as a function of varying baseline response of the sensor in the absence of oxygen. The baseline response of the sensor in the absence of oxygen is determined in a nitrogen atmosphere.

In a number of embodiments, the ambient current output of the sensor is less than 300 μA, optionally, less than 150 μA, or optionally less than 30 μA.

In another aspect, a gas detection device includes a control system including a processor system and a memory system, and an electrochemical sensor analytically responsive to oxygen including a housing comprising a capillary via which gas diffuses into the housing from an environment, a working electrode within the housing in operative connection with the control system, a counter electrode within the housing in operative connection with the control system, and an electrolyte within the housing in ionic contact with the working electrode and the counter electrode. The control system is configured, via execution of software stored in the memory system by the processor system, to apply an electrical signal to the electrochemical sensor to generate a current flow between the working electrode and the counter electrode via the electrolyte, to measure a parameter of a response of the electrochemical sensor to the electric signal, to compare the measured parameter to a predetermined characterization of the parameter, wherein the predetermined characterization of the parameter provides a relationship between the parameter and a response of the electrochemical sensor over varying state of the electrochemical sensor, and to determine an output value of the gas detection device from the analytical response electrochemical sensor in sensing oxygen in the ambient environment and the comparison of the measured parameter to the predetermined characterization.

The control system may be further configured to operate the electrochemical sensor in a sensing mode wherein the output signal, which is representative of a concentration of oxygen in the environment, is generated, and to operate the electrochemical sensor in an interrogation mode during which the electrochemical sensor is electronically interrogated by applying the electrical signal to the electrochemical sensor, the measured parameter being measured in the interrogation mode. The predetermined characterization may be saved in the memory system at the time of manufacture.

In a number of embodiments, the parameter is a chronoamperometric parameter. The parameter may, for example, be, or be a function of, maximum peak value, area under the curve, minimum peak value, peak-to-peak value and reverse area under the curve of the response of the electrochemical sensor to the electric signal applied in the interrogation mode.

In a number of embodiments, an ambient current output of the sensor is less than 300 μA, optionally, less than 150 μA, or optionally less than 30 μA.

In a further aspect, a method of characterizing changes in response of an electrochemical sensor over varying state of the electrochemical sensor (wherein the electrochemical sensor is capillary limited and analytically responsive to oxygen, and the electrochemical sensor include a housing comprising a capillary via which gas diffuses into the housing from an environment, a working electrode within the housing, a counter electrode within the housing, and an electrolyte within the housing in ionic contact with the working electrode and the counter electrode) includes determining a predetermined characterization providing a relationship between a parameter measured upon application of an electrical signal to the electrochemical sensor to generate a current flow between the working electrode and the counter electrode via the electrolyte and a response of the electrochemical sensor over varying state of the electrochemical sensor.

The devices, systems, and methods hereof along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates schematically a cross-sectional view of a capillary-limited electrochemical gas sensor hereof.

FIG. 1B illustrates schematically a perspective cutaway view of a capillary-limited electrochemical gas sensor hereof.

FIG. 1C illustrates schematically an enlarged view of the capillary inlet of the capillary-limited electrochemical gas sensor of FIG. 1A.

FIG. 2 illustrates a representative example of sensor response (for two difference working electrode sizes) to a pulse test hereof in which energy to the working electrode is changed resulting in a change of current passing therethrough.

FIG. 3 illustrates studies of representative capillary-limited electrochemical gas sensors hereof showing changes in AUC, DeltaPeak, and MaxPeak parameters as a result of a pulse test hereof, demonstrating that such parameters include the same of very similar analytical information.

FIG. 4A illustrates the dependency of nitrogen baseline (capacitive) current on change in the parameter AUC.

FIG. 4B illustrates a measurement of the nitrogen baseline on baseline sensor current in the absence of oxygen.

FIG. 5 illustrates ambient sensor output correlated linearly with nitrogen baseline changes, demonstrating that the oxygen influence is constant over the evaluated environmental change.

FIG. 6 illustrates the ambient output of the sensor is correlated to the change in chronoamperometric pulse test parameter.

FIG. 7 illustrates that the chronoamperometric pulse test parameter allows prediction of sensor output in relevant values.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As set forth above, response measurements resulting from the evaluation of the correlation of sensor life and health analytical data and sensor sensitivity via electronic interrogation have not been sufficiently well correlated in the past to allow sufficiently accurate correction of output values or readings of a capillary-limited electrochemical oxygen sensor. As used herein, “sensor sensitivity” refers generally to the ratio between the change in output signal (for example, current or voltage) and a measured property.

Without limitation to any underlying mechanism, studies of devices, systems, and sensor hereof, have indicated that electrochemical parameters measured in an electronic interrogation in which energy to the working electrode of a sensor is changed are not correlated with changes in sensor sensitivity. Studies of electrochemical, capillary-limited oxygen sensors hereof have indicated that the sensor signal height or amplitude is determined only by the capillary and diffusion characteristics of the gas inlet of the sensor. Mainly, such characteristics include the effective capillary diameter and length and the carrier gas background. Once again, without limitation to any mechanism, the present studies indicate that sensor nitrogen baseline (or baseline without the influence of oxygen) is dependent on changes in sensor state arising, for example, from environmental conditions such as humidity development and sensor history. The overall sensor signal or response is a sum of both the sensor baseline without oxygen (sometimes referred to herein as the nitrogen baseline) and the response to oxygen. The present studies further demonstrate that a characterization of sensor state and corresponding response of the sensor may be determined as a function of one or more parameters determined in an electronic interrogation in which an electrical signal is applied to the electrochemical sensor to cause a current to flow between the working electrode and the counter electrode via the electrolyte. Such a characterization or predetermined characterization may, for example, provide a relationship between one or more parameters and a response of the electrochemical sensor over varying state of the electrochemical sensor. The response of the electrochemical sensor measured in the predetermined characterization of the electrochemical sensor may include only the baseline output/response of the electrochemical sensor in the absence of oxygen or be the ambient output/response (including both the baseline response of the electrochemical sensor in the absence of oxygen and the response to oxygen).

During the determination of the predetermined characterization, a variation in state of the electrochemical sensor (for example, a variation electrolyte concentration and local water content at the working and reference electrode of the electrochemical sensor system) or a variation in state of one or more representative, like electrochemical sensors may, for example, be induced by exposure over time to an ambient environmental condition which is different from a “standard” ambient environmental condition at a calibration. The parameter or parameters being characterized and sensor response may be measured at different times (for example, periodically) over the time of exposure to the nonstandard ambient condition. It is also possible to induce changes in state without long-term exposure to an ambient environmental condition different from a calibration ambient environmental condition. For example, one may manually or automatically change electrolyte concentration/water content in a studied electrochemical sensor and measure the one or more parameters after each such change while, for example, maintaining ambient environmental conditions the same or approximately the same as the ambient environmental conditions at calibration.

The output value or sensor reading of an electrochemical, capillary limited oxygen sensor (for example, provided as vol % O₂) may be determined from two sensor parameters. In that regard, the sensor current during operation in ambient air at 20.8 vol % O₂ (I_{amb} or the current output at ambient conditions) is first determined. The output current during operation in a known calibration gas concentration is also determined. The calibration gas may, for example, be pure nitrogen with 0.0 vol % O₂ content as described above. As also described above, the current output during operation in nitrogen atmosphere is referred to herein as nitrogen baseline (I_{nitrogen}) of the sensor. The sensor sensitivity may be calculated from the above two values as follows:

$$sensitivity [ A / Vol \% ] = ( {I}_{amb} - {I}_{nitrogen} ) [ A ] / 20.8 [ Vol \% ]$$

To calculate the sensitivity, other pairs or multiple pairs of O₂ concentrations may be used. The choice of concentrations may be determined by the desired measurement range and background of the technical application. From the above equations on sensitivity, the sensor output reading of an electrochemical, capillary-limited oxygen sensor may be calculated from the following equation:

$$sensor reading [ Vol \% ] = ( actual / measured sensor current - {I}_{nitrogen} ) [ A ] / sensitivity [ A / Vol \% ]$$

Currently, the sensor nitrogen baseline is treated as a constant value. However, the presents studies indicate that the sensor output reading or value can be influenced by changes in both sensitivity and nitrogen baseline. Regardless of the underlying mechanism, the devices, systems, and methods hereof provide a predetermined characterization which provides a relationship between a parameter determined in an electronic interrogation of the sensor and a response of the electrochemical sensor over varying sensor state. The response of the electrochemical sensor in the predetermined characterization may be determined as a change in response of the electrochemical sensor compared to a response at a value determined at an initial or calibrated state under defined conditions. Without limitation to any mechanism, the varying state/response of the electrochemical sensor may be associated with change in electrolyte concentration and local water content at, for example, the working and the reference electrode of the electrochemical sensor system. The predetermined characterization may be used in determining the sensor output reading or value, which may be considered a corrected sensor output reading or value. In that regard, one may determine an output value of the gas detection device from (i) a measured output signal (for example, a current signal) in response to exposure to gas in an ambient environment and (ii) a comparison of a parameter measured in an electronic interrogation (contemporaneous to the measure output signal) to the predetermined characterization of that parameter. The implementation of a correction methodology in the devices, systems, and methods hereof, which provides improved accuracy in sensor output readings or values, can be accomplished in various ways. In that regard, a change in sensor output reading (associated with the measured parameter in an electronic interrogation in reference to the predetermined characterization of the parameter) may, for example, be implemented into an algorithm changing the overall sensitivity of the sensor, changing the nitrogen baseline, changing a general correction factor, etc. The implementation of the sensor output determination can be independent from the actual naming of the variables described above.

Currently available methodologies for determining life and health of capillary-limited electrochemical gas sensors for oxygen have attempted to determine either the signal height or the overall signal. In a number of embodiments, the devices, systems, and methods hereof provide for determination or characterization of the sensor state (and associated response) as a function of a parameter determined in an electronic interrogation to predict the overall signal through addition of the constant signal height provided by the presence of atmospheric oxygen. As described above, the characterization of the sensor state/associated response as a function of a parameter determined in an electronic interrogation may, for example, be determined by dependency of the parameter upon varying sensor state of the electrochemical sensor over time. In a number of studies hereof, the variation in sensor state was induced by exposure to predetermined environmental conditions over time. Such environmental conditions may vary significantly (in, for example, temperature and/or relative humidity) from environmental conditions during a calibration.

Prior to the present studies, studies of capillary-limited oxygen sensors (which were typically operated under relatively high power) have indicated that ambient output signal height or amplitude was independent of environmental conditions such as humidity. Given the relatively small effect of changes in capacitive or non-faradaic current associated with changes in nitrogen baseline resulting from changing state of the electrochemical sensor arising, for example, from environmental conditions as described herein, the signal-to-noise ratio of the electronics of such sensors was typically insufficient to observe and characterize the phenomenon.

As used herein and in the appended claims, the singular forms “a,” “an”, and “the” include plural references unless the context clearly dictates otherwise. Thus, for example, reference to “a parameter” includes a plurality of such parameters and equivalents thereof known to those skilled in the art, and so forth, and reference to “the parameter” is a reference to one or more such parameters and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”, “circuitry” or “circuit,” as used herein include, but is not limited to, hardware, firmware, software, or combinations of each to perform a function(s) or an action(s). For example, based on a desired feature or need. a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

In the case of application of an electrical signal to a working electrode of an electrochemical gas sensor in an electronic interrogation, a response may, for example, be measured in the form of (or as a function of) (i) a maximum peak value (MaxPeak), which is the maximum current observed upon the application of the potential pulse; (ii) an area under the curve (AUC), which is the integrated current response of the working electrode after the application of the potential pulse (this is equivalent to the charging response of the sensor; (iii) minimum peak value (minPeak), which is the minimum current obtained upon removal or reversal of the potential pulse, ordinarily as the difference in current observed immediately after and immediately before the removal or reversal of the potential pulse, though it can also be tabulated and used as the difference between the minimum current and the baseline; (iv) peak-to-peak value (PP), which is the algebraic difference between the maximum and minimum observed currents; and (v) reverse area under the curve (rAUC), or, more accurately, the area under the reverse curve, which is the charging current obtained by integrating the current response after the removal or reversal of the potential pulse. In a number of embodiments hereof, one or more of these parameter values determined during a period of time in which the sensor is used to monitor analyte concentration may, for example, be compared to values of the parameter in a predetermined characterization in which sensor response (for example, as represented by changes in the nitrogen baseline) is related to the parameter over varying sensor state. The predetermined characterization may, for example, be saved in memory of a device including the sensor at the time of manufacture. In a test or pulse cycle of an interrogation mode, the energy to the working electrode is increased or decreased for a period of time (for example, via a change in current or voltage), which is typically a brief period of time, and the resultant response is measured.

Electronic interrogations may, for example, be of fairly short duration to minimize the amount of time a sensor is offline to conduct sensor testing diagnostics (that is, during a sensor electronic interrogation cycle). In a number of embodiments, electronic interrogation may allow for a return to a normal (gas sensing) mode operation for the electrochemical sensors hereof that is under 10 seconds, under 5 seconds even under 1 second, or even under 0.5 seconds. Devices, systems and methods for electronic interrogation of sensor may allow an instrument including one or more sensors to remain “online.” Moreover, such devices, systems and method may also provide for active, automatic sensor status monitoring as a background operation, without the requirement of user initiation. The frequency of the electronic interrogations may vary. Providing for sensor interrogation at a frequency of, for example, several times an hour can provide for nearly constant sensor life and health status monitoring.

In the case of a gas sensor, it is desirable that detection should occur in the gas phase, or at a phase boundary. Generally, this observation indicates that the speed of the sensor will be limited only by the rate of gas phase diffusion of target gas molecules to the sensor. For purposes of limiting sensor output, gas sensors such as electrochemical gas sensors may, for example, be permeation- or diffusion-controlled or -limited, wherein a permeable membrane is used to limit diffusion of the target gas into the sensor, or capillary-controlled or-limited, wherein a capillary inlet is used to limit diffusion of the target gas into the sensor.

In that regard, in an electrochemical gas sensor, the gas to be measured (sometimes referred to as the target gas or analyte gas) typically passes from the surrounding atmosphere or environment into a sensor housing through, for example, a gas porous or gas permeable membrane or through a capillary inlet to a first electrode or working electrode (sometimes called a sensing electrode) at which a chemical reaction occurs. A complementary chemical reaction occurs at a second electrode known as a counter electrode (or an auxiliary electrode). The electrochemical sensor produces an analytical signal via the generation of a current arising directly from the oxidation or reduction of the analyte gas (that is, the gas to be detected) at the working electrode. A comprehensive discussion of electrochemical gas sensors is also provided in Cao, Z. and Stetter, J. R., “The Properties and Applications of Amperometric Gas Sensors,” Electroanalysis, 4(3), 253 (1992), the disclosure of which is incorporated herein by reference.

The working and counter electrode combination produces an electrical signal that is (1) related to the concentration of the analyte gas and (2) sufficiently strong to provide a signal-to-noise ratio suitable to distinguish between concentration levels of the analyte gas over the entire range of interest. In other words, the current flow between the working electrode and the counter electrode must be measurably proportional to the concentration of the analyte gas over the concentration range of interest.

In addition to a working electrode and a counter electrode, an electrochemical sensor often includes a third electrode, commonly referred to as a reference electrode. A reference electrode is used to maintain the working electrode at a known voltage or potential. The reference electrode should be physically and chemically stable in the electrolyte.

Electrical connection between the working electrode and the counter electrode is maintained through the electrolyte. Functions of the electrolyte include: (1) to efficiently carry the ionic current; (2) to solubilize the analyte gas; (3) to support both the counter and the working electrode reactions; and (4) to form a stable reference potential with the reference electrode. Criteria for an electrolyte may, for example, include the following: (1) electrochemical inertness; (2) ionic conductivity; (3) chemical inertness; (4) temperature stability; (5) low cost; (6) low toxicity; (7) low flammability; and (8) appropriate viscosity.

In general, the electrodes of an electrochemical cell provide a surface at which an oxidation or a reduction (a redox) reaction occurs to provide a mechanism whereby the ionic conduction of the electrolyte solution is coupled with the electron conduction of the electrode to provide a complete circuit for a current. The measurable current arising from the cell reactions of the electrochemical cell is directly proportional to the extent of reaction occurring at the electrode. Preferably, therefore, a high reaction rate is maintained in the electrochemical cell. For this reason, the counter electrode and/or the working electrode of the electrochemical cell generally include an appropriate electrocatalyst on the surface thereof to support the reaction rate.

As a result of electrostatic forces, the volume of solution very close to the working electrode surface is a very highly ordered structure. This structure is important to understanding electrode processes. The volume of solution very close to the electrode surface is variously referred to as the diffusion layer, diffuse layer, and or the Helmholtz layer or plane.

The magnitudes of the resistance and capacitance present in an electrochemical cell are a result of the nature and identities of the materials used in its fabrication. The resistance of the electrolyte is a result of the number and types of ions dissolved in the solvent. The capacitance of the electrode is primarily a function of the effective surface area of the electrocatalyst. In an ideal world, these quantities are invariant. However, the solution resistance in an amperometric gas sensor that utilizes an aqueous (water-based) electrolyte may change, for example, as a result of exposure to different ambient relative humidity levels. As water transpires from the sensor, the chemical concentration of the ionic electrolyte increases. This concentration change can lead to increases or decreases in the resistivity of the electrolyte, depending on the actual electrolyte used.

Moreover, even for substances normally thought of as insoluble in a particular solvent, there is a small, but finite concentration of the substance in the solvent. For example, there is a very small, but finite concentration of metal from the electrodes dissolved in the electrolyte of an electrochemical sensor. This small concentration of dissolved metal is constantly in flux. That is, metal atoms are constantly dissolving from the electrode and then replating somewhere else. The net effect of this process is to decrease the effective surface area of the electrode. This has the effect of lowering the sensor capacitance over time. Both of the above-described effects have the net effect of changing the sensor output over its lifetime.

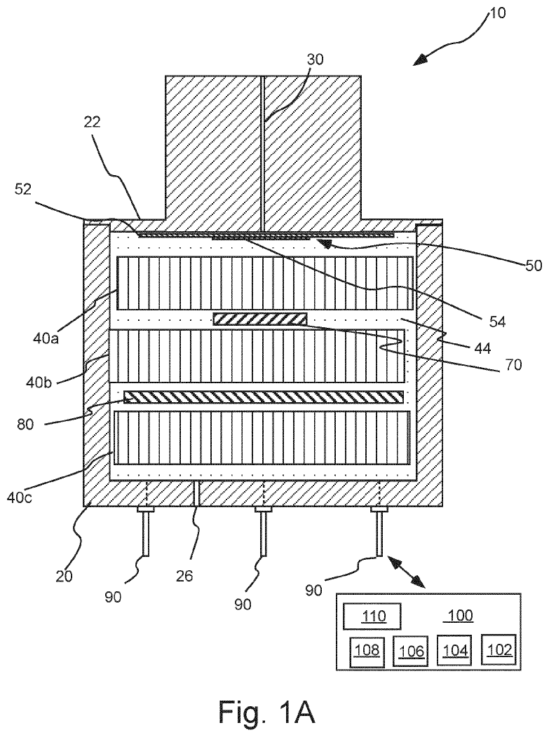

FIGS. 1A and 1B illustrate schematic diagrams of a representative embodiment of a capillary-limited electrochemical sensor 10 of the devices, systems and methods hereof. Sensor 10 includes a housing 20 having a gas inlet 30 in the form of a capillary for entry of one or more target gases or analyte gases into sensor 10. As the name indicates, a capillary-limited sensor such as sensor 10 uses a very small inlet hole 30 (that is, a capillary) with a common or typical aspect ratio (length: diameter or 1:d) of approximately 100:1 (see, for example, FIG. 1C, which illustrates an axial and a radial cross-sectional view of inlet 30 and a cylindrical portion of housing 20 therearound).

In FIG. 1C, p₂ is the partial pressure of the target gas outside inlet 30, p₁ is the partial pressure of target gas at an inside opening of inlet 30, c₂ is the concentration of the target gas outside inlet 30 and c₁ is the concentration of target gas at an inside opening of inlet 30 (or the surface working electrode 50, which is essentially zero). What is often referred to as “normal capillary diffusion” is actually a special case of Graham's law of effusion. See, for example, Barrow, G. M.: Physical Chemistry, 4th edition. New York NY: McGraw Hill (1979). In general, “diffusion” refers to the bulk flow of a gas from a region of higher pressure (or partial pressure) or higher concentration through a porous wall or tube of very small diameter, to a region of lower pressure or lower concentration, respectively. “Effusion” refers to a process of movement resulting from molecular, rather than bulk, flow through the orifice or membrane.

Capillary-limited oxygen or O₂ sensors are the dominant O₂ sensor in the marketplace. This dominance is mostly likely because many performance standards are written in terms of volume-percent (vol-%) O₂ concentration. A capillary-limited O₂ sensor measures vol-% O₂ without dependence upon O₂ partial pressure (which varies with total atmospheric pressure even at a constant vol-% O₂ concentration). In other words, the capillary-limited sensor simply responds to vol-% target gas in a sample regardless of pressure. The output of a capillary sensor is provided by the following equation:

$${i}_{lim} = 2.12 {D}_{0} \frac{{d}^{2}}{l} {( T )}^{1 / 2} ( \frac{{v}_{1}}{V} ) .$$

This equation indicates that the sensor output iₗᵢₘ is directly dependent on the dimensions of the capillary d²/l. D₀ is the target gas (for example, O₂) diffusion coefficient. Moreover, the sensor output change according to the square root of temperature (T^{1/2}, or about 0.17% per degree C.). Further, v₁/V (or the volume of the target gas vi divided by the volume V of the test environment being sensed by the sensor) is the volume fraction of the target gas in the test environment (for example, the volume of O₂ in a test atmosphere.

In a number of embodiments, electrolyte saturated wick materials 40a, 40b and 40c may separate working electrode 50 from a reference electrode 70 and a counter electrode 80 within sensor 10 and/or provide ionic conduction therebetween via the electrolyte 44 within housing 20 and absorbed within wick materials 40a, 40b and 40c. Electronic circuitry 100 as known in the art is provided, for example, to maintain a desired potential difference between working electrode 50 and reference electrode 70, to vary or pulse the potential difference as described herein, and to process an output signal from sensor 10. The sensor electrodes are placed in connection with electrical circuitry 100 via connectors 90 which provide conductive electrical conductivity/connectivity through housing 20.

In the illustrated embodiment, working electrode 50 may be formed by, for example, depositing a first layer of electrocatalyst 54 on a gas diffusion membrane 52 (using, for example, catalyst deposition techniques known in the sensor arts). While sensor 10 may include gas diffusion membrane 52 behind capillary inlet 30, unlike the case of a permeation-or diffusion-limited sensor, diffusion through gas diffusion membrane 52 is not rate limiting. Membrane 52 serves to retain electrolyte 44 within housing 20 and to support electrocatalytic layer/surface 54 within sensor 10. Gas readily transfers or transports (via, for example, diffusion) through diffusion membrane 52, but electrolyte 44 does not readily transfer or transport therethrough. Diffusion membrane 52 of working electrode 50 may be attached (for example, via heat sealing) to an inner surface of a top, cap or lid 22 of housing 20. A representative working electrode 50 may, for example, include platinum or platinum dispersed on carbon as electrocatalyst layer 54. An acidic electrolyte such as H₂SO₄ may, for example, be used.

Electronic circuitry 100 include, for example, a processor or controller system 102 including one or more processors or microprocessors to control various aspects of the operation of sensor 10. A memory system 104 may be placed in operative or communicative connection with processor system 102 and may store software for control, measurement and/or analysis in sensor 10. A user interface system 106 (including, for example, a display, speaker etc.) may also be placed in operative or communicative connection with processor system 102. A communication system 108 such as a transceiver may be placed in operative or communicative connection with processor system 102 for wired and/or wireless communication. A power source 110 (for example, a battery system and/or line power) may provide power for electronic circuitry 100.

As illustrated in FIGS. 1A and 1B, in a number of embodiments, a vent 26 is formed in sensor housing 20 which is in gaseous communication with counter electrode 80. Vent 26 allows O₂ produced at counter electrode 80 to escape housing 20. The amount of O₂ produced is quite small (only a few nanoliters per second). However, over the lifetime of sensor 10, the produced O₂ can become quite significant. Unless sensor 10 is efficiently vented, pressure will increase in sensor housing 20 and either perturb the sensor signal or cause electrolyte leakage.

In a number of studies hereof, sensor based upon XCELL® oxygen sensor available from MSA Safety Incorporated of Cranberry Township, Pennsylvania were used to characterize capillary-limited oxygens sensors. Oxygen sensors with 12 μm capillary diameter were tested over 6 months in different storage conditions. After a calibration in ambient conditions, in one study group, sensors were placed into chambers with conditions of 25° C. and 10% relative humidity (r. h.). The ambient conditions used during calibration were approximately 22° C. and a relative humidity in the range of 40 to 50%. In another study group, after calibration in ambient conditions, sensors were placed in chambers with conditions of 25° C. and 85% r. h. All sensors were tested regularly on nitrogen baseline (that is, a baseline in the absence of oxygen or 0 vol % oxygen output), 20.8 vol % oxygen output, and 10.4 vol % oxygen output. After about 2 months of storage in such chambers, the conditions were reversed and sensors formerly maintained under dry conditions were placed in humid conditions and vice versa. Each sensor test was accompanied with a chronoamperometric pulse or interrogation test. Chronoamperometry refers to an electrochemical technique in which the potential of the working electrode is changed (for example, stepped) and the resulting current is monitored as a function of time. The electronic interrogations hereof occur without the application of a test gas having a known concentration of the analyte gas or a simulant therefor to the sensor from a container. In a number of studies, the pulse or interrogation test included a 10 mV bias change for 1 sec. The result of the voltage change is a current curve from which the representative parameters baseline (Baseline), the maximum current (MaxPeak), area under curve (AUC) and the signal height between baseline and MaxPeak (DeltaPeak) were determined. All values were corrected for their baseline value.

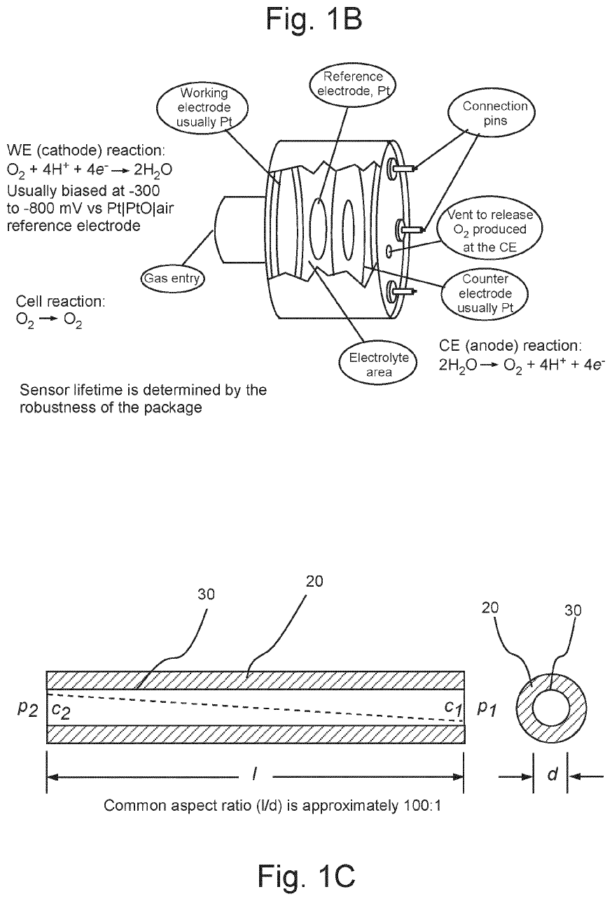

To study the influence of working electrode size, the tested sensors included two different sizes of working electrodes which had the same specific surface area. One set of sensors had standard-sized working electrodes (0.19 inch or 4.83 mm diameter) while a second group of sensors had larger working electrodes (0.312 in or 7.92 mm diameter). The results of the studies of working electrode size demonstrated two distinct groups in pulse test response as illustrated, for example, in FIG. 2. In that regard, working electrode size was found to change the absolute values of the current response during pulse testing, but not the gas response. The gas response current is determined only by the capillary characteristics. The working electrode size influences the pulse pattern, but the conclusions drawn from the chronoamperometric data is independent of working electrode size. Such studies demonstrate that the system response evaluation hereof is universal, and devices, systems, and methods hereof allow the prediction of system behavior over a wide variety of electrochemical O₂ sensing embodiments.

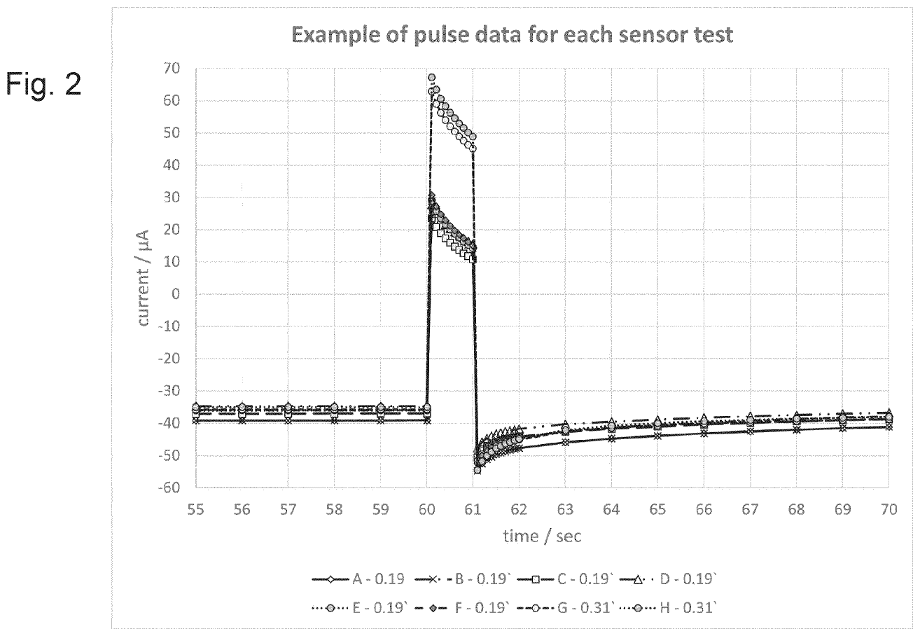

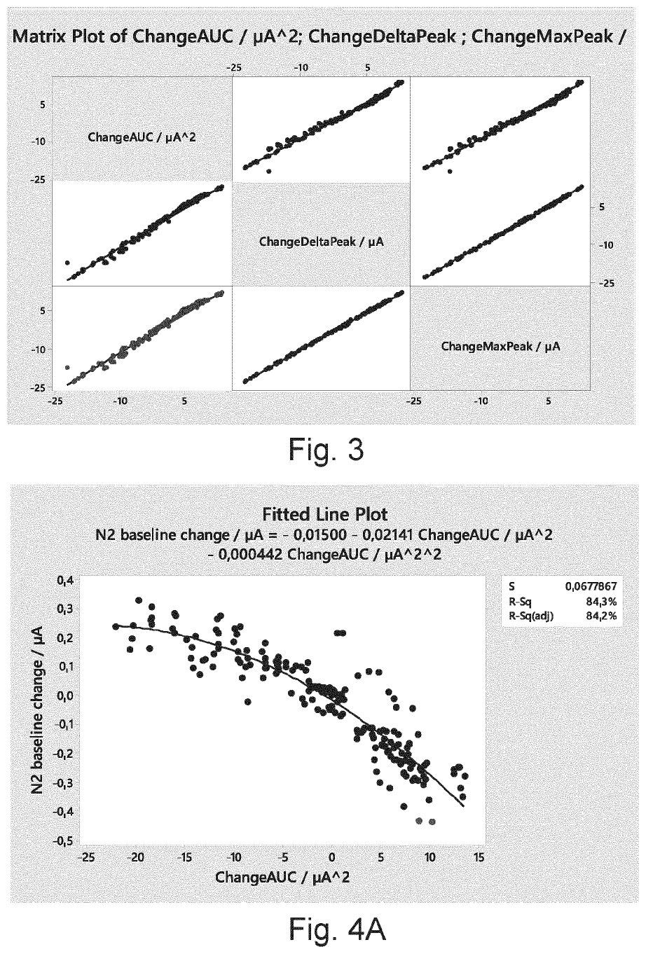

The predictive evaluation of the pulse test or electronic interrogation data includes two steps and highlights newly discovered aspects of the system behavior of capillary-limited oxygen sensors. In general, all pulse parameters are strongly interconnected and the prediction is independent of the choice of parameter as illustrated, for example, in the matrix plot of FIG. 3. As described above, the evaluation of all environmental dependent data have not previously shown a detectable, direct connection between the pulse data and the sensor ambient output in sensors which were typically operated at relatively high power. However, in a number of embodiments of the devices, systems, and methods hereof, the nitrogen baseline of the sensor, in dependency of sensor state as changed with environmental conditions, is correlated with, for example, chronoamperometric pulse data and/or one or more parameters determined via other electrochemical characterization(s). The nitrogen baseline current in absence of oxygen represents the background current of the electrochemical system of the sensor. That parameter depends on the environmental conditions/history and changes in a predictable manner with the chronoamperometric response as illustrated, for example, in FIG. 4A.

FIG. 4B illustrates the difference between a nitrogen baseline (that is, a baseline in the absence of oxygen) and an ambient baseline (that is, a baseline in 20.8 vol % oxygen or O₂, which is the standard oxygen concentration in ambient air). In the absence of oxygen (for example, in an atmosphere of pure nitrogen or N₂), the current through the sensor is a nearly completely a capacitive current which is driven by double-layer processes within the sensor. In the presence of oxygen, the sensor signal includes a combination of the capacitive “nitrogen baseline” signal and the signal arising from the faradaic reduction current. As shown in the studies hereof, the capacitive, nitrogen baseline current detectably changes with change in environmental factors (for example, humidity).

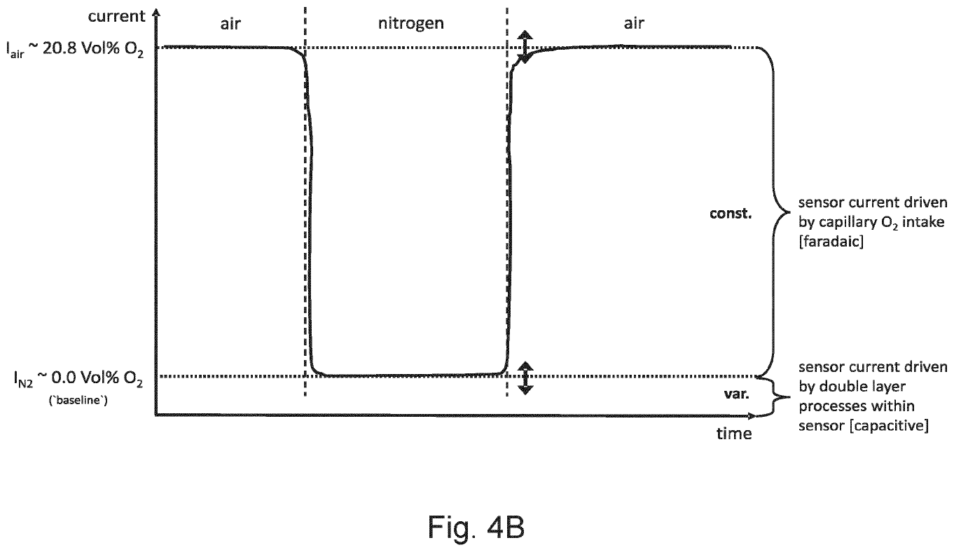

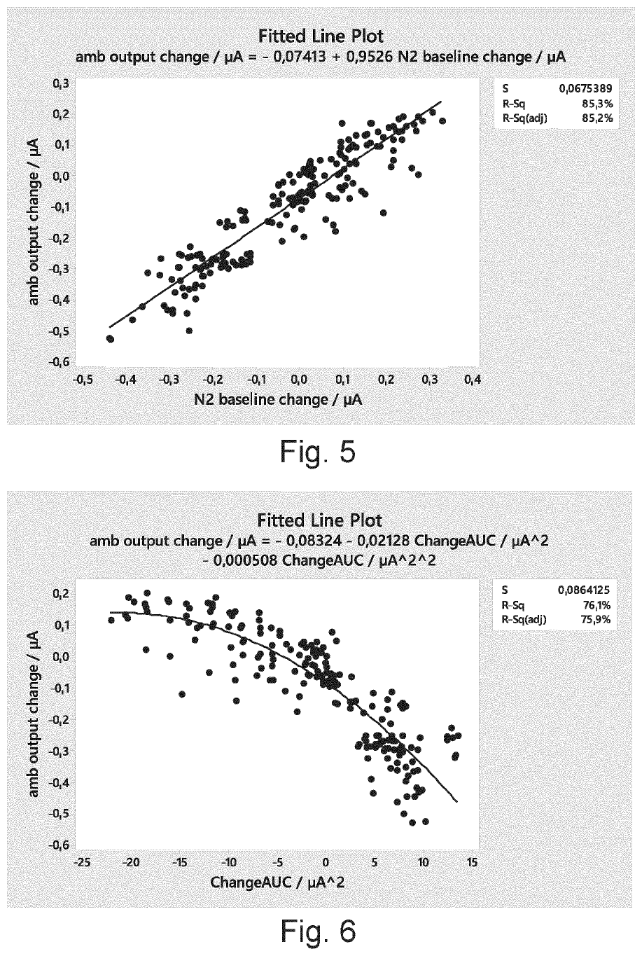

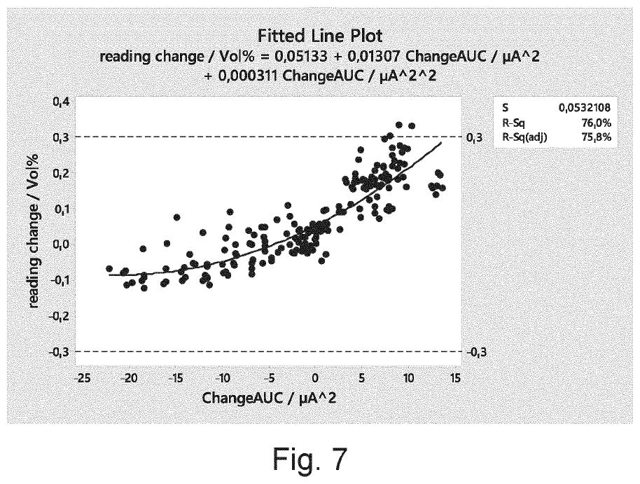

As set forth above, the actual sensor reading or sensor output is a combination of its nitrogen baseline and the added signal resulting from the influence of incoming oxygen. The influence of oxygen entering the sensor is capillary driven only and is constant across humidity changes which influence the nitrogen baseline (see, for example, FIG. 5). Without limitation to any mechanism, it is likely that the electrolyte concentration and local water content at the working and reference electrode of the electrochemical sensor system influence the nitrogen baseline over humidity and environmental conditions. The correlation is also visible in the ambient output of the sensors studied herein, which is the combination of baseline and oxygen influence (see, for example, FIG. 6). Because the actual sensor reading is determined only by its actual current output, the correlations hereof allow the prediction of the sensor reading change as a function of the chronoamperometric or other electrochemical evaluation (see, for example, FIG. 7, which illustrates the sensor output change (vol %) as a function of change in the parameter AUC (μA²)). Output changes refers to the difference between a measured valued of a parameter and the value determined at calibration of the sensor at ambient conditions described above. The value of reading change of 0.0 Vol % (no signal change) thus occurs at a parameter output change of 0.0.

The characterization of chronoamperometric data (and/or other parameters measured after inducing current flow) over a range of sensor state (for example, wherein change in sensor state induced by environmental condition such as relative humidity, temperature, etc.) as, for example, described in certain embodiments hereof, provides a predetermined relationship between such parameters and change in the state of the sensor. Such a change in state may, for example, be demonstrated by a change in the nitrogen baseline response (and thereby the sensor output) of the sensors hereof in response to change in sensor stated induced by environmental conditions/history. The determined relationship between one or more measured electrochemical parameters and sensor output/response over varying sensor state may be stored in the memory system of the sensor (for example, as an equation or algorithm, or in a look-up table). Suitable measured parameters are not limited to chronoamperometric parameters, but can be parameters measured in any type of electrochemical measurement in which an electrical signal is applied to an electrochemical sensor to generate a current flow between the working electrode of the electrochemical sensor and the counter electrode via the electrolyte (for example, in electrochemical impedance spectroscopy of EIS, in cyclic voltammetry or CV, etc.). One or more algorithms may further be stored in the memory system for execution by the processor system to correct analyte gas (oxygen) concentration output of the sensor based upon one or more electrochemical parameters measured near in time or at the same time of the sensor measurement of analyte gas concentration (for example, within a day thereof, within an hour thereof, within minutes thereof, or within seconds thereof) giving rise to the sensor output. In that regard, the one or more parameters such as chronoamperometric parameters are measured over the period of time (subsequent to manufacture) that the sensor is in operation to measure the target analyte (oxygen). As set forth above, in a number of embodiments, the choice of parameter is not of importance as the various parameters provide similar or the same analytical data. Referring, for example, to FIG. 7, data of output reading change as a function of a chronoamperometric or other parameter may be stored in memory (for example, at the time of manufacture—as, for example, identified over a range of sensor state) and applied to sensor output on the basis of a contemporaneous measurement of the chronoamperometric parameter and/or another electrochemical parameter. In that regard, a sensor device displays a certain output value which may be calculated using a stored sensitivity and nitrogen baseline as described above. The predetermined characterization data determined in the methodologies hereof may be used in the calculation and/or to adjust output values determined in the calculation to provide a more accurate output value.

A predetermined characterization or relationship between sensor state/output reading change and chronoamperometric or other electrochemical parameter(s) may, for example, be determined for an individual sensor or for one or more sensors representative of a class of similar or like sensors. As used herein, the term “similar sensor” or “like sensor” refers to sensor having the same or similar design parameters (for example, the same or similar electrodes, electrolytes etc.). As described herein, the predetermined characterization may be determined on the basis of change in nitrogen baseline response or ambient response over induced changes in sensor state. Alternatively, one or more mathematical models may be used to determine the predetermined characterization based, for example, upon characterization of design parameters of a sensor or class of sensors and relationships developed from theory and/or experimental data.

The devices, systems, and methods hereof thus provide for the prediction/correction of the reading of a capillary-limited oxygen sensor over relevant ranges and practically available environmental conditions. In addition to improving maintenance of sensor output accuracy, this functionality can be used for a variety of improvements in oxygen sensing technology including, for example, predictive maintenance, increased worker safety, decrease of sensor downtime, and providing sensor operation over a wider range of environmental conditions.

Recent trends in the gas sensor art have been toward sensors requiring less power. In sensors operated under relatively high power, the faradaic current is relatively large compared to environmentally driven changes in the capacitive current. As described above, it is more difficult in a sensor operated under relatively high power, to detect changes in capacitive current, which are a very small portion of the ambient output current. With decreasing sensor current, typically associated with smaller capillaries in capillary-limited gas sensors, the relative influence of the capacitive, nitrogen baseline increases. Changes in the capacitive baseline become sufficiently large that changes in sensor output as a result of environmental changes are significant. In the case of a standard, relatively high-power capillary-limited oxygen sensors, the ambient output may, for example, be in the range of approximately 300 to approximately 250 μA. In a number of embodiments of sensors hereof, the ambient current output is no greater than 300 μA, no greater than 250 μA, no greater than 150 μA, no greater than 100 μA, no greater than 80 μA, or no greater than 30 μA. In general, the lower the ambient current output of the sensor, the greater the percentage of capacitive current arising from environmental change in that output. Although, the effect of change in capacitive current with environmental conditions is observable in the ambient output of a sensor as illustrated in FIG. 6, a predetermined characterization of that change measured over a range of sensor state by studying nitrogen baseline change may provide better characterization results given the greater percentage of total signal associated with changes in capacitive current in the case of measurement of the nitrogen baseline signal.

The devices, systems, and methods hereof may be used in connection with relatively high-powered capillary-limited oxygen sensors. However, the electronics (for example, a high-power, low-noise potentiostat) of such sensors must provide suitable signal-to-noise ratio to determine the effect of change in capacitive current described herein accurately and reproducibly.

In summary, in the devices, systems, and methods hereof a characterization of a change in response of a sensor (for example, as evidenced by changes in the nitrogen baseline value) over change in sensor state (which may, for example, arise from a change in or history of environmental condition) is determined. In that regard, a characterization or predetermined characterization of a parameter determined in an electronic interrogation provides a relationship between the parameter and a response of the electrochemical sensor over varying state of the electrochemical sensor. The electrochemical parameter is measured in, for example, a pulse test/electronic interrogation in which energy to the working electrode of the electrochemical sensor is changed. The predetermined characterization is used to modify or correct the sensor output readings or values based upon a relation or comparison of the predetermined characterization to a contemporaneous measurement of the electrochemical parameter. Sensor output readings or values may thus be adjusted or corrected for changes in sensor state resulting, for example, from the influence of changes in environmental condition or history.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A method of operating a gas detection device including a capillary-limited electrochemical sensor which is analytically responsive to oxygen, the electrochemical sensor comprising a housing comprising a capillary via which gas diffuses into the housing from an environment, a working electrode within the housing, a counter electrode within the housing, and an electrolyte within the housing in ionic contact with the working electrode and the counter electrode, comprising:
applying an electrical signal to the electrochemical sensor to generate a current flow between the working electrode and the counter electrode via the electrolyte;
measuring a parameter of a response of the electrochemical sensor to the electric signal,
comparing the measured parameter to a predetermined characterization of the parameter, wherein the predetermined characterization of the parameter provides a relationship between the parameter and a response of the electrochemical sensor over varying state of the electrochemical sensor; and
determining an output value of the gas detection device from an output signal electrochemical sensor in sensing oxygen in the ambient environment and the comparison of the measured parameter to the predetermined characterization.

2. The method of claim 1, further comprising:
operating the electrochemical sensor in a sensing mode, wherein the output signal, which is representative of a concentration of oxygen in the environment, is generated; and
operating the electrochemical sensor in an interrogation mode, during which the electrochemical sensor is electronically interrogated by applying the electrical signal to the electrochemical sensor, the measured parameter being measure in the interrogation mode.

3. The method of claim 1, wherein the gas detection device further includes a control system including a processor system and a memory system, wherein the working electrode is in operative connection with the control system and the counter electrode is in operative connection with the control system and the memory system comprising one or more algorithms stored thereof and executable by the processor system to carry out one or more the actions of the method.

4. The method of claim 2 wherein the gas detection device further includes a control system including a processor system and a memory system. wherein the working electrode is in operative connection with the control system and the counter electrode is in operative connection with the control system, the memory system comprising one or more algorithms stored thereof and executable by the processor system to carry out one or more of the actions of the method.

5. The method of claim 1, wherein the parameter is a chronoamperometric parameter.

6. The method of claim 5, wherein the parameter is or is a function of at least one of maximum peak value, area under the curve, minimum peak value, peak-to-peak value and reverse area under the curve of the response of the electrochemical sensor to the electric signal applied in the interrogation mode.

7. The method of claim 1, wherein the predetermined characterization is determined over a varying state of the electrochemical sensor.

8. The method of claim 7, wherein the varying state of the electrochemical sensor is induced by environmental condition.

9. The method of claim 7, wherein the predetermined characterization is determined as a function of varying baseline response of the electrochemical sensor in the absence of oxygen.

10. The method of claim 9, wherein the baseline response of the electrochemical sensor in the absence of oxygen is determined in a nitrogen atmosphere.

11. The method of claim 1, wherein an ambient current output of the electrochemical sensor is less than 300 μA.

12. A gas detection device, comprising:
a control system including a processor system and a memory system, and
an electrochemical sensor analytically responsive to oxygen comprising a housing comprising a capillary via which gas diffuses into the housing from an environment, a working electrode within the housing in operative connection with the control system, a counter electrode within the housing in operative connection with the control system, and an electrolyte within the housing in ionic contact with the working electrode and the counter electrode,
the control system being configured, via execution of software stored in the memory system by the processor system, to apply an electrical signal to the electrochemical sensor to generate a current flow between the working electrode and the counter electrode via the electrolyte; to measure a parameter of a response of the electrochemical sensor to the electric signal, to compare the measured parameter to a predetermined characterization of the parameter, wherein the predetermined characterization of the parameter provides a relationship between the parameter and a response of the electrochemical sensor over varying state of the electrochemical sensor; and to determine an output value of the gas detection device from the analytical response electrochemical sensor in sensing oxygen in the ambient environment and the comparison of the measured parameter to the predetermined characterization.

13. The gas detection device of claim 12, wherein the control system is further configured to operate the electrochemical sensor in a sensing mode, wherein the output signal, which is representative of a concentration of oxygen in the environment, is generated, and to operate the electrochemical sensor in an interrogation mode, during which the electrochemical sensor is electronically interrogated by applying the electrical signal to the electrochemical sensor, the measured parameter being measured in the interrogation mode.

14. The gas detection device of claim 12, wherein the predetermined characterization is saved in the memory system at the time of manufacture.

15. The gas detection device of claim 12, wherein the parameter is a chronoamperometric parameter.

16. The gas detection device of claim 15 wherein the parameter is or is a function of at least one of maximum peak value, area under the curve, minimum peak value, peak-to-peak value and reverse area under the curve of the response of the electrochemical sensor to the electric signal applied in the interrogation mode.

17. The gas detection device of claim 12, wherein an ambient current output of the electrochemical sensor is less than 300 μA.

18. A method of characterizing changes in response of an electrochemical sensor over varying state of the electrochemical sensor, the electrochemical sensor being capillary limited and analytically responsive to oxygen, the electrochemical sensor comprising a housing comprising a capillary via which gas diffuses into the housing from an environment, a working electrode within the housing, a counter electrode within the housing, and an electrolyte within the housing in ionic contact with the working electrode and the counter electrode, the method comprising: determining a predetermined characterization providing a relationship between a parameter measured upon application of an electrical signal to the electrochemical sensor to generate a current flow between the working electrode and the counter electrode via the electrolyte and a response of the electrochemical sensor over varying state of the electrochemical sensor.

19. The method of claim 4 wherein the predetermined characterization is saved in the memory system at the time of manufacture.
